(19) United States
(12) Patent Application Publication
(10) Pub. No.: US 2006/0185679 A1
(43) Pub. Date: Aug. 24, 2006

(54) Lose at the source plate (L.A.T.S.Plate)

(76) Inventors: Sun Hye Costigan, Novato, CA (US); Benthram Thomas Costigan, Novato, CA (US)

Correspondence Address: SUN H. COSTIGAN, DDS, 210 SADDLE WOOD DRIVE, NOVATO, CA, 94945, US

(21) Appl. No.: 11/062,504

(22) Filed: Feb. 22, 2005

(51) Int. Cl. A61F 5/56; A61C 5/14

(52) U.S. Cl. 128/848; 128/859; 128/861; 128/862

(57) ABSTRACT

This invention is an intra-oral device (appliance) that will assist a user with weight loss by physiologically stimulating appetite suppression. This intra-oral device is a thin, molded acrylic or plastic plate that extends beyond the junction of the hard and soft palate (vibrating line) and is retained in place by wire clasps around selected teeth. The intra-oral appliance resembles a tooth retainer worn by post-orthodontic patients (after braces are removed); however, the shape is distinctly different, with a portion of the acrylic plate extended beyond the junction of hard and soft palate. When the plate extends past the vibrating line into the soft palate area, it impinges on an area of the oropharynx where a partial-gag reflex is located. The Vagus Nerve (Cranial Nerve X) is primarily responsible for upward movement of the soft palate and it is known to be related to appetite and anxiety centers in the medulla of the brain. This device works by stimulating this sensitive area (the Vagus Nerve) as a person attempts to swallow a large mass of food and gives the physiologic response of pre-gag or partial-gag reflex. This in turn causes a non-voluntary, physiologic avoidance response, and the user temporarily loses the desire to eat large boluses of food, thus suppressing appetite. It does not inhibit or prohibit swallowing but causes the user to resort to tiny pieces of food, mashed food, or liquids in small quantities. It also inhibits a person's ability to guzzle or gorge foods in a hurried manner. This translates into slower, more incremental eating behaviors and results in more rapid satiation because of the slowed ingestion. Similar intra-oral devices called stay-plates are in common usage in the dental profession for the temporary replacement of one or more extracted teeth. These appliances are safe to wear because they are too large to be swallowed or aspirated to the lungs and have been in common usage for more than a century. This device resembles a toothless stay-plate (with retaining clasps) that has an extended posterior palatal protrusion over the junction of the hard and soft palate. This is the first intra-oral appliance invention that works to stimulate physiological reflexes that are well-known and documented phenomena. All other intra-oral weight loss devices have relied in one way or another upon physical obstruction of the oral cavity or alteration of the amount of space for which food passes. This invention does not rely on reduction of available space within the mouth, and on the contrary, is a very thin plate which does not interfere with breathing or normal mastication. There are no detrimental effects on the teeth, oral tissues, or on a person's overall health if used as recommended and under proper supervision. It is recommended that the user consult with their physician and dentist, prior to, during, and following the use of this appliance to be sure their general health and oral health are in good order. Several modifications of the appliance design uses wires extending from the appliance or natural teeth to accomplish the stimulation behind the junction of the hard and soft palate (vibrating line).

### BACKGROUND OF THE INVENTION

1. Field of the Invention

The invention relates to intra-oral devices (appliances) that are used to assist users with weight control measures. The invention differs significantly from other patented devices that are recommended for weight control. The invention relies on mechanical stimulation of known receptor sites of the Vagus Nerve (Cranial Nerve Number 10), located on the soft palate just posterior to the vibrating line (junction of hard and soft palate). These well-known and documented receptor sites perform safety functions for the body in the form of a reflex arc. Reflexes are non-voluntary and happen automatically. The neuronal arc begins at the receptor sites on the soft palate and travels to the medulla area of the brain. The medulla is part of the primitive brain stem that is not involved with cognitive function, but has ties to emotional behaviors and autonomic responses. The reflex arc returns to innervate the muscles of the soft palate (the velum) and also the musculature of the oropharynx. The soft palate (velum) moves upward to close the opening of the nasal passages when food is being swallowed. If the receptors are triggered, the reflex arc is designed to forcefully expel the contents of the oropharynx and protect the body from ingesting a potentially harmful object or material attempting to enter the gastrointestinal or brachial tracts. There are many levels of responses inherent to the stimulation of this reflex. Mild stimulation can result in a quick cough. Mild stimulation can also cause a partial-gag reaction similar to ‘rolling of the stomach.’ Medium to high stimulation can cause forceful coughing, choking, and full-gagging reaction. All levels of stimulation of the reflex produce an avoidance reaction, anxiety, and suppression of appetite, to varying degrees depending on the level of the stimulus. This invention seeks to provoke a mild stimulation of the area in such a way as to suppress appetite without evolving too strong of a gag reflex. As will be discussed on other sections of this application, other patented intra-oral devices or appliances have attempted to limit the volume of space available in the mouth for food to pass. Some devices have attempted to disrupt the efficiency of chewing, interfere with the user's ability to ingest food, or in some other way be an obstruction within the oral cavity. This invention is unique in that it does not act in an obstructive manner, but rather relies on the body's normal physiological processes to gain the desired effect (weight loss through appetite suppression).

2. Related Art

There are many methods that one can utilize to assist in weight loss and control. People that want to lose weight should first consult with their physician regarding possible systemic diseases or ailments that may be contributing to excess weight. Once cleared or monitored for system diseases, the universal wisdom for losing body weight is through diet and exercise. By limiting caloric intake and burning more calories than ingested will result in weight loss in healthy people. If recommended by a competent physician or dietitian, many diet and exercise plans are effective ways to lose weight and are the first choice to accomplish weight control.

However, many ‘diet and exercise’ individuals have a self-control problem. That is, they lack the will-power to adhere to diet and exercise recommendations in a manner that gives them the results they seek. Many people lose weight during early phases of diet and exercise, but later gain it back when their will-power fails them and they resume their previous behaviors. People that have tried numerous weight loss schemes and repeatedly failed to achieve a favorable long-term result have asked the question of what other alternatives are available. Unfortunately, many individuals have been lured into ‘fad’ diets and are on the constant search for the next trendy diet plan for their never ending quest for weight-loss programs.

Individuals that know they have a lack of will-power have sought drastic and intrusive measures to lose weight, such as having their teeth wired shut or having surgery on their stomach or intestines. Such invasive procedures such as ‘stomach stapling’ or teeth wiring are unpleasant alternatives because these measures are extreme.

A barrage of intra-oral devices have been patented over the years that claim usefulness for weight control. In 1999, Gustafson (U.S. Pat. No. 5,924,422) described an invention that constricts the volume of the user's oral cavity and forces them to slow their intake of food. This device essentially makes the palatal area of your mouth so thick that one cannot eat efficiently when the obstructive device is in place. This device relies on the fact that by inhibiting the rate of food ingestion, one becomes more quickly satiated. Gustafson does not, however, claim to suppress appetite with this device. It is possible that many obese people are used to eating to engorgement, and are not simply satisfied when their blood sugar elevates to a point of removing hunger sensations.

In 1989, Bessler (U.S. Pat. No. 4,883,072) described elastic devices used in the mouth and lips to inhibit the uptake of food without interfering with breathing and speech. As with the Gustafson device described above, it is another obstructive device that causes the wearer to limit food ingestion capabilities, but it does not claim appetite suppression.

In 1988, Brown, et al (U.S. Pat. No. 4,738,259) described a dental appliance that interfered with mastication (chewing) and claimed to be a device that would assist the user with weight control. Again, the primary action of this device was to obstruct food ingestion. In this case, the obstruction was directed at the efficiency of the chewing apparatus.

In 1984, Brown, et al (U.S. Pat. No. 4,471,771) claimed a weight loss device which was essentially a sieve that was worn in the mouth. This sieve-like appliance acted as a filter in which ground foods and liquids could pass and solid food items would not fit through the gateway.

In 1991, Stubbs (U.S. Pat. No. 5,052,410) claimed an intra-oral device that had tabs extending between the cheek and gums which could be used for weight control and tobacco cessation. The device appears to be obstructive in nature, but the inventor also described an increased saliva production mechanism which contributed to its action.

In 1988, Knoderer (U.S. Pat. No. 4,727,867) introduced an intra-oral device that interfered with a person's normal masticatory (chewing) function. The invention claimed to inhibit the user's lateral jaw movements and permit only up-and-down, vertical chewing ability. This device obstructed the efficiency of the chewing mechanism and slowed ingestion.

All of the aforementioned inventions employ various methods of obstruction or disruption of a person's ability to imbibe or ingest food. Whether by reducing the available space in the mouth, physically interfering with mastication (chewing), or by introducing an intra-oral obstacle, these devices force a slowed ingestion for the users. All the cited references take advantage of the well-known and commonly accepted phenomenon that eating slower allows individuals to lose hunger sensations (i.e., reach satiation). The physiological mechanism for this relates to digestion and increasing blood-sugars. Increasing glucose and other carbohydrates (and sugars) in the blood stream shuts down the hunger centers in the brain.

One problem with obesity is that some people overeat to the level of engorgement, and not simply stop eating when hunger sensations have dissipated. Unfortunately, many people do not stop eating once they no longer are hungry. It is one thing to remove hunger sensations, but it is a distinctly different concept dealing with eating until one is “full” or “stuffed.” Even if the hunger sensations are turned off, if people are accustomed to engorge themselves, they will not feel ‘satiated’ until their stomachs are distended to the point of being “full.” Eating slowly by itself may not help people that are used to the feeling of being “full.”

The obstructive devices cited in the references do not claim appetite suppression and this is a necessary component for maximum effectiveness. These obstructive devices have no effect on an individual's desire to eat past the point of hunger dissipation.

### SUMMARY OF THE INVENTION

This invention is unique to the field of related art in that its primary mode of action is to stimulate appetite suppression. The invention does so by taking advantage of a well-understood reflex arc that exists on the soft palate just behind the vibrating line (junction of hard palate and soft palate). Generically, we are referring to the gag reflex, which is a well-documented function of the Vagus Nerve (Cranial Nerve Number X). Depending on the level and/or force of the stimulation, the gag reflex can be a mild sensation or a full-blown choking, coughing, or vomiting reaction. This invention attempts to stimulate what we have called a pre-gag or partial-gag reflex. The idea is to have users receive enough stimulation to change their normal eating behavior, but no so strong as to induce a major gag reflex. Anyone that has ever experienced a forceful gag reaction knows well that it has a profound effect on anxiety and your appetite. Imagine the last time you gagged on a fish bone or got something “caught in your throat.” All of a sudden, you are not hungry any more, and much of the time the experience has simply, “ruined your whole meal” and you walk away from the table, having “lost your appetite.” In many cases, people retain a long-standing fear of whatever has caused them this discomfort, and will refrain from that food in the future (e.g., I don't like fish anymore because of the bone that made me gag). Essentially, you will develop a new perspective on food and have behavioral changes that persist to the future. Once new “habits” develop, the user has now adjusted to a new approach to eating.

When people use this invention, they will have difficulty gulping or gorging foods with the appliance in place. If they used to love eating large pieces of steak and lots of it, their experience will be drastically different when they sense a “rolling of the stomach” caused by stimulation of the partial-gag reflex. By using this invention, gorging down large pieces of steak very quickly is no longer a desirable option. Once they experience the “rolling stomach,” all of a sudden their normal behavior is associated with an uncomfortable sensation. This results in effective appetite suppression because your mind now has a different perspective on gorging food or gobbling large bites. Therefore, this invention has the unique effect of causing changes in eating behavior by stimulating normal physiological mechanisms in the body. Changing eating behavior is essential to successful long term management of weight.

While wearing the appliance, users cannot eat large boluses of food in a hurried manner. Therefore, a secondary effect of the invention is eating slower and using smaller pieces of food. As discussed in the Related Art section, eating slower and with smaller bites helps you lose the hunger sensation more quickly. But, the slowed eating effect is not the primary mechanism of action for this invention.

The users are advised to use the appliance in the following manner:

Upon rising in the morning, the user will brush and floss their teeth and follow other recommendations given by their dentist or dental hygienist. After oral hygiene is complete, the appliance is inserted into the mouth. As an added incentive not to remove the appliance throughout the day, the user is advised to place several drops of denture adhesive on the inside of the appliance. That way, it is sealed into place with the sticky adhesive used by denture wearers. The user is less likely to remove the appliance because of the adhesive. The user has the appliance in place during breakfast, lunch, and dinner. Before retiring or going to sleep, the user would remove the appliance and thoroughly brush and clean it. Also, the user would brush, floss, rinse, or employ other oral hygiene procedures recommended by their dental professionals. The appliance can soak in water or denture cleaner during the night, and the user gets a break from wearing it, much the same as patients wearing a dental prosthesis.

Users should have routine visits to their dentist and primary care physician. Weight loss and nutrition should be monitored by trained health professionals. Physicians or dentists will be able to recommend dietary supplements or other nutritional adjuncts to make sure users are proceeding in a safe and effective manner.

### MANUFACTURE AND DESCRIPTION OF THE DRAWINGS

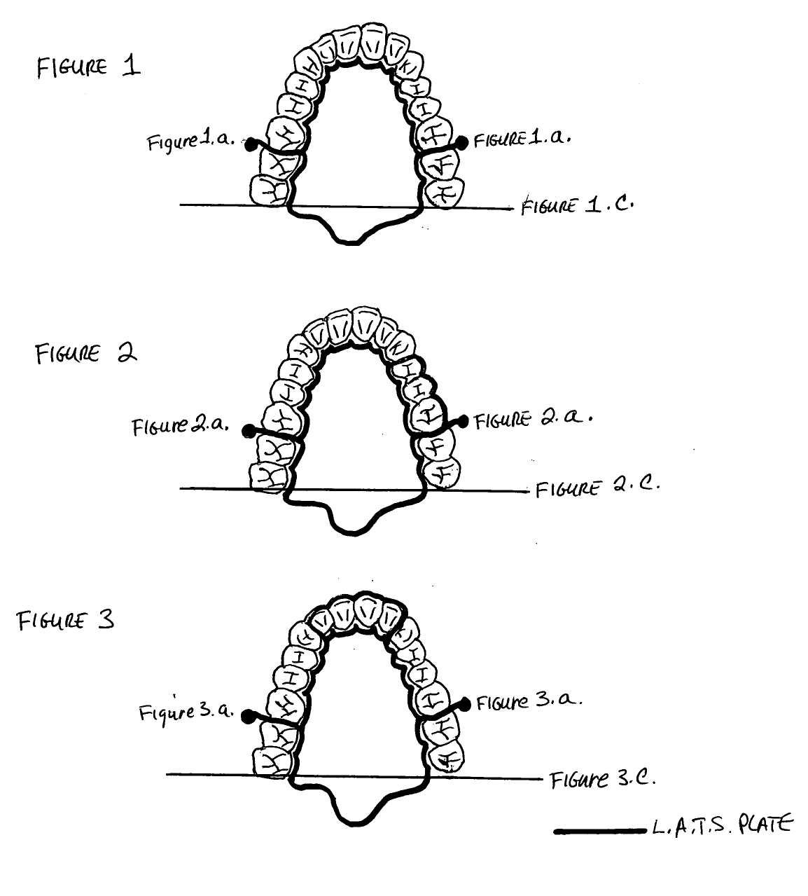

Drawings of the Lose At The Source (L.A.T.S.) Plate are presented on page 2 of this section as FIGS. 1, 2 and 3. This unique invention differs from a standard dental appliance in that this palatal plate is designed to be manufactured to extend somewhat beyond the border between the hard and soft palate. This extension can be seen as a tab extending beyond the vibrating line (FIGS. 1c, 2c and 3c) shown in FIGS. 1, 2 and 3. The device is described in significant detail in that portion of this U.S. patent application subtitled Abstract. The L.A.T.S. plate can be manufactured without fabricated teeth (FIG. 1) or with fabricated teeth (FIGS. 2 and 3); can be integrated into existing denture, orthodontic or prosthodontic appliances (FIG. 3); does not cause orthodontic tooth movement and is retained by suction, clasps around natural teeth, and/or denture adhesives. The L.A.T.S. plate is manufactured as follows:
a. A dentist/denturist/trained professional conducts an oral examination of the patient to determine the pathology of the hard and soft palate and records the position of the vibrating line.
b. An impression of the full upper arch is taken to include a portion of the upper palate extending beyond the vibrating line. This impression is made using alginate, hydro-colloid impression material, or other dental impression materials.
c. The alginate or other material impression is then poured using plaster or dental stones (Yellowstone) or other denture modeling material.
d. An outline of the palate and gingival margins is made (see FIG. 1b)
e. The post dam is manufactured to be one or more millimeters longer than the established vibrating line (FIGS. 1c, 2c and 3c) and the outline is carved lightly to provide post dam margin and adaptation to the tissues.
f. The upper palatal impression is then waxed up using standard wax up techniques used in the manufacture of dental prostheses (stayplates or dentures).
g. The plate is then manufactured by a dental laboratory or other appropriate facility using an acrylic, plastic or other denture material, fitted with standard ball clasps or other retaining devices (FIGS. 1a, 2a, and 3a) and is cured under pressure.
h. The complete plate is then polished and finish product delivered.
i. An alternative to the fabrication of a removable appliance would be to have a fixed orthodontic or bendable wire put in place directly in the patient's mouth. The orthodontic or bendable wire would have the same post dam configuration as the L.A.T.S. Plate and mimic the action of the removable appliance (extension beyond the vibrating line). The wires would be fixed to the teeth using orthodontic brackets, orthodontic bands, or other hardware that anchor the wires safely in place.

The completed L.A.T.S. plate is then delivered to the patient by the dentist/denturist or other trained professional. The plate is then fitted to the patient to insure comfort and utility. The patient is then instructed to wear the device daily and to remove and clean the plate at bedtime.

I claim:

1. An intra-oral appliance used to suppress appetite and assist users in losing body weight. The appliance consists of:
An intra-oral appliance positioned against the roof of the mouth (palate) that extends beyond the junction of the hard and soft palate (vibrating line) that activates the pre-gag or partial-gag (or in extreme situations, full-gag) reflex when a large bolus of food is introduced into the mouth.

2. An appliance according to claim 1 that can be fabricated by taking an impression (mold) of the upper (maxillary) teeth and palatal structures and designed to be retained in the mouth by clasps around selected teeth.

3. An appliance according to claim 1 that can also be a modified full denture, partial denture, stayplate, or any other dental prosthetic device used in the mouth to replace missing teeth, gums, or bone structures.

4. An appliance according to claim 1 that can also be a modified orthodontic retainer used to brace teeth following orthodontic treatment.

5. An appliance according to claim 1 that can be orthodontic wires retained by bands around teeth or brackets affixed to teeth that extend beyond the junction of the hard palate and soft palate to stimulate the partial-gag reflex.

6. An appliance that is fabricated from any combination of materials, such as acrylic, plastic, cast metal, molded metal, orthodontic wires, clasps, or other materials suitable for use in the mouth which is designed to have an extension that crosses the junction of the hard and soft palate (vibrating line) for the purpose of stimulating the pre-gag or partial-gag (or full-gag) reflex, in an effort to suppress appetite. The appliance can be removable from the mouth or fixed in place with brackets or bands around selected teeth. In the case of a removable appliance, selected teeth are fitted with clasps or retaining wires that are bended around the neck of the teeth to hold it in place.

7. An appliance as in claims 1-6 that is fabricated for the user's intent to lose weight by stimulating a sensitive area on the soft palate behind the vibrating line (junction of hard palate and soft palate) which suppresses appetite.

8. An appliance as in claims 1-6 that also causes users to eat more slowly and with smaller pieces of food, mashed food, or liquefied foods, for the purpose of becoming more quickly satiated through slower ingestion.

9. An appliance as in claims 1-6, which if a removable design, would be retained in the mouth through inter-digitations of the appliance around the palatal side of selected teeth of the user, and also by use of clasps or wires that extend to the facial side of selected teeth (side facing the cheek, also called the buccal aspect of the tooth). The removable appliance design also utilizes rest seats on selected teeth which are areas that seat the appliance and ‘click it’ into a definitive resting place. This appliance is designed specifically not to put lateral or undesirable forces on teeth that would cause the teeth to shift.
